SEQUENCE LISTING (4 sequences)

SEQ ID NO: 1 - DNA, 20 bp, Artificial Sequence
  Synthetic (forward primer)
ctggcatgaacatgaccctg

SEQ ID NO: 2 - DNA, 21 bp, Artificial Sequence
  Synthetic (reverse primer)
ctgacctaaagccacctcctt

SEQ ID NO: 3 - DNA, 20 bp, Artificial Sequence
  Synthetic (forward primer)
agccaggaacgtactggtga

SEQ ID NO: 4 - DNA, 20 bp, Artificial Sequence
  Synthetic (reverse primer)
gcctccttctgcatggtatt

(19) United States
(12) Patent Application Publication
(10) Pub. No.: US 2015/0037801 A1
(43) Pub. Date: Feb. 5, 2015

(54) METHOD OF AMPLIFYING NUCLEIC ACID

(71) Applicant: Samsung Electronics Co., Ltd., Suwon-si (KR)

(72) Inventors: Dong-hyun PARK, Chuncheon-si (KR); Yeon-jeong Kim, Yongin-si (KR); Kyung-yeon Han, Seoul (KR)

(73) Assignee: Samsung Electronics Co., Ltd., Suwon-si (KR)

(21) Appl. No.: 14/180,931

(22) Filed: Feb. 14, 2014

(30) Foreign Application Priority Data
Aug. 2, 2013 (KR) 10-2013-0092249

(51) Int. Cl. C12Q 1/68

(52) U.S. Cl. CPC C12Q 1/6806; C12Q 1/6851
USPC 435/6.12

(57) ABSTRACT

Provided is a method of amplifying a nucleic acid of a cell involving applying ultrasonic waves to lyse a cell bound to a solid support to provide a cell lysate comprising a nucleic acid; separating the cell lysate from the solid support; adding a protease to the cell lysate; and amplifying the nucleic acid.

### CROSS-REFERENCE TO RELATED APPLICATIONS

This application claims the benefit of Korean Patent Application No. 10-2013-0092249, filed on Aug. 2, 2013, in the Korean Intellectual Property Office, the entire disclosure of which is incorporated herein by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 1,031 Bytes ASCII (Text) file named “715710_ST25.TXT,” created on Feb. 11, 2014.

### BACKGROUND

1. Field

The present disclosure relates to a method of amplifying a nucleic acid by lysing a cell specifically bound to a solid support via cytolysis.

2. Description of the Related Art

The technology of nucleic acid amplification has been used for fast and accurate diagnosis of various biological samples, and its importance has gradually increased. In general, the nucleic acid of an isolated cell can be amplified by PCR by simply lysing the cell without a purification step.

However, when a cell is attached to an antibody-bound particle, PCR amplification may not be efficiently performed using only conventional cell lysis without a specific purification step.

Accordingly, there is a need for the development of a method of efficient amplification of a nucleic acid in a cell which is attached to an antibody-bound particle using simple cell lysis.

### SUMMARY OF THE INVENTION

According to an aspect of the present invention, there is provided a method of amplifying a nucleic acid in a cell specifically bound to a solid support.

According to an aspect of the present invention, there is provided a method of amplifying a nucleic acid by applying ultrasonic waves to a cell specifically bound to a solid support to lyse the cell and provide a cell lysate; separating the cell lysate from the solid support; adding a protease to the separated cell lysate and incubating the protease and cell lysate; and amplifying a nucleic acid from the cell lysate incubated with the protease using a nucleic acid polymerase.

### DETAILED DESCRIPTION OF THE INVENTION

The method includes applying ultrasonic waves to lyse a cell bound to a solid support applying ultrasonic waves to lyse a cell. The solid support may be any solid support to which cells may be bound. For instance, the solid support may be a bead or packing material for a column typically used for separating biological components. The solid support may have any suitable shape, such as a spherical, polygonal, plate, or linear form, or a combination thereof. The solid support may be made of any suitable material, such as polystyrene, polypropylene, a magnetic particle, or a combination thereof.

The cell is bound to the solid support via a material that specifically binds to the cell. The material which specifically binds to a cell may be, for example, a surface protein, a lipid, or a material that binds to a sugar. The material which can specifically bind to a cell may be, for example, a material that binds a protein, a substrate of an enzyme, a co-enzyme, a regulating factor, a material that specifically binds to a receptor, a lectin, a sugar, a glycoprotein, an antigen, an antibody, an antigen-binding fragment, a hormone, a neurotransmitter, phospholipid-binding protein, a protein including a pleckstrin homology (PH) domain, a cholesterol-binding protein, or a combination thereof. The antigen-binding fragment includes an antigen-binding domain and may be, for example, a single-domain antibody, Fab, Fab′, or scFv. The material may bind specifically to a cell by intervening into a lipid bilayer of the cell. The material that can intervene into the lipid bilayer of the cell may be a lipophilic moiety, an amphipathic moiety, an amphoteric ion moiety, or a combination thereof. The lipophilic moiety may be, for example, fatty acid, sterol, or glyceride. The amphipathic moiety may be, for example, phospholipid or sphingolipid. The amphoteric ion moiety may be, for example, sulfobetaine, carboxybetaine, or phosphorylcholine.

The cell may be any desired target cell to be separated from a biological sample from a subject. The subject may be a mammal, such as a human. The biological sample refers to a sample obtained from a biological organism. The biological sample may include, for example, urine, mucus, saliva, tears, blood, blood plasma, blood serum, sputum, spinal fluid, pleural fluid, nipple aspirate, lymph, respiratory tract fluid, serous fluid, urogenital fluid, breast milk, lymph secretion, semen, cerebrospinal fluid, body fluid in organs, ascites, fluid from a cystic tumor, amniotic fluid, or a combination thereof. The cell may include nerve cells, epithelial cells, reproductive cells, immune cells, muscle cells, cancer cells, or a combination thereof. The cancer cells may be, for example, circulating tumor cells, cancer stem cells, or general cancer cells. The cell may be a cell of a cancer or tumor selected from the group consisting of bladder cancer, breast cancer, cervical cancer, cholangiocarcinoma, colorectal cancer, endometrial cancer, esophageal cancer, stomach cancer, head and neck cancer, kidney cancer, liver cancer, lung cancer, nasopharyngeal cancer, ovary cancer, pancreatic cancer, gallbladder cancer, pancreatic cancer, thyroid cancer, osteosarcoma, rhabdomyosarcoma, synovial sarcoma, Kaposi's sarcoma, leiomyosarcoma, malignant fibrous histiocytoma, fibrosarcoma, adult T-cell leukemia, lymphoma, multiple myeloma, glioblastoma/astrocytoma, malignant melanoma, mesothelioma, and Wilm's tumor.

The ultrasonic wave is a sound wave equal to or greater than about 16 kHz of frequency, or greater than about 20 kHz, for example, from about 16 kHz to about 20 kHz, about 20 kHz to about 2.0 MHz, about 100 kHz to about 2.0 MHz, about 500 kHz to about 2.0 MHz, about 1.0 MHz to about 2.0 MHz, about 1.2 MHz to about 1.8 MHz, or about 1.4 MHz to about 1.6 MHz. The ultrasonic wave may be applied to the cell solution or suspension within a chamber disposed in an ultrasonic water bath. The ultrasonic wave may be applied, for example, at room temperature or at about 4° C. The duration of application of the ultrasonic wave may be adjusted appropriately as known in the art. The application of the ultrasonic wave can destroy a cell membrane, thereby lysing the cell.

The cell lysis may be performed in the presence of a solution containing a cation. The cation refers to an atom or atomic group charged electropositively. The cation may be a monovalent or divalent cation. The cation may be K⁺, Na⁺, Mg²⁺, or a combination thereof. The cation may be in the range of about 50 mM to about 200 mM, about 50 mM to about 180 mM, about 50 mM to about 160 mM, about 50 mM to about 140 mM, about 50 mM to about 120 mM, or about 50 mM to about 100 mM. The solution may further include a surfactant. The surfactant may be an anionic surfactant, a cationic surfactant, an amphoteric surfactant, or a non-ionic surfactant. The non-ionic surfactant may be polyoxyethylene glycol alkyl ether, polyoxypropylene glycol alkyl ether, glucoside alkyl ether, polyoxyethylene glycol octylphenol ether, polyoxyethylene glycol alkylphenol ether, glycerol alkyl ester, polysorbate, sorbitan alkyl ester, cocamide, dodecylmethylamine oxide, polyethoxylated tallow amine (POEA), or a combination thereof, for example, TRITON™ X-100 or TWEEN® 20.

According to an exemplary embodiment of the present invention, the method includes separating a cell lysate from the solid support.

The cell lysate may include a nucleic acid. The nucleic acid may be, for example, genomic DNA, messenger RNA (mRNA), ribosomal RNA (rRNA), transfer RNA (tRNA), or fragments thereof.

The separation of the cell lysate from the solid support may be a separation by centrifugation, a separation by a magnet, a separation by filtration, or a combination thereof.

The separation of the cell lysate from the solid support may include washing the solid support. The washing may be performed, for example, at room temperature or at about 4° C.

According to another exemplary embodiment of the present invention, the method includes adding a protease to the separated product and incubating the same.

The protease may be serine protease, threonine protease, cysteine protease, aspartate protease, glutamic acid protease, metalloprotease, or a combination thereof. The serine protease may be, for example, proteinase K.

The protease is incubated with the cell lysateat about 15° C. to about 65° C., for example, in the range of about 20° C. to about 60° C., about 25° C. to about 55° C., or about 30° C. to about 50° C. The incubation may be conducted for any suitable length of time, for instance, as long as a protease maintains its activity or the lysate contains reactants for the protease.

According to a further exemplary embodiment of the present invention, the method includes amplifying the nucleic acid using a nucleic acid polymerase.

The nucleic acid polymerase may be DNA polymerase, RNA polymerase, reverse transcriptase, or a combination thereof. DNA polymerase may be, for example, Taq DNA polymerase, Pfx DNA polymerase, Tfi DNA polymerase, Tth DNA polymerase, Tfl DNA polymerase, hot start polymerase, or a combination thereof. The RNA polymerase may be, for example, T7 RNA polymerase or SP6 RNA polymerase. The reverse transcriptase may be, for example, Moloney Murine Leukemia Virus (MMLV) reverse transcriptase, Human Immunodeficiency Virus (HIV) reverse transcriptase, or Avian Myeloblastosis Virus (AMV) reverse transcriptase.

As used herein, the term “amplification” refers to an increase in copy number of a nucleic acid, and it includes the generation of DNA from RNA. The amplification may be performed by any known method. The amplification method may require thermal cycling or may be performed at isothermal conditions. For example, the amplification may include polymerase chain reaction (PCR), nucleic acid sequence-based amplification (NASBA), ligase chain reaction (LCR), strand displacement amplification (SDA), rolling circle amplification (RCA), or a combination thereof. The amplification method may also include a method of RNA amplification, for example, reverse transcription (RT) or RT-PCR. As used herein, the term “PCR” refers to a method of amplifying a target nucleic acid from a primer pair which specifically binds to the target nucleic acid by using a polymerase. For example, the amplification of a nucleic acid by PCR repeats a cycle of denaturation, annealing, and elongation. As used herein, the term “annealing” may be replaced with the term “hybridization”. Additionally, the amplification may be DNA amplification or RNA amplification. The nucleic acid amplification may be, for example, a real-time nucleic acid amplification. As used herein, the expression “Real-Time PCR (RT-PCR)” refers to a method which observes the increase in PCR product per each cycle and analyzes a sample based on the detection and quantification of a fluorescent material which reacts with the PCR product.

The nucleic acid amplification may be whole genomic nucleic acid amplification or targeted nucleic acid amplification.

According to an exemplary embodiment of the present invention, the method of the nucleic acid amplification enables the amplification of a nucleic acid of a cell specifically bound to a solid support without separating the cell from the solid support.

### EXAMPLES

Hereinafter, the present disclosure is further illustrated by the following examples and comparative examples. However, it shall be understood that these examples are only used to specifically set forth the present disclosure, and they are not limitative in any form. As used herein, the term “and/or” includes any and all a combination of one or more of the associated listed items.

### Example 1

### Preparation of a Sample

Genomic DNA isolated from leukocytes of a healthy person was prepared using a QIAamp DNA mini kit (Qiagen). HCC827 cells were prepared and incubated with beads (DynaBeads Epithelial Enrich from Life Technologies) bound anti-EpCAM antibody to capture the cells on the beads.

### Example 2

### PCR of Cell Lysates obtained by Lysing Cells Captured on Beads

The sample prepared in Example 1 was incubated and a quantitative polymerase chain reaction (qPCR) was performed without an additional purification step.

60 pg of the genomic DNA, 10 HCC827 cells, and 10 HCC827 cells captured on the beads prepared in Example 1 were incubated at 55° C. for 1 hour in the presence of a cell lysis buffer 2 (50 mM KCl, 1.5 mM MgCl₂, and 10 mM Tris-HCl (pH8.3)) and/or 1 μg of proteinase K (Sigma). In addition, the 10 HCC827 cells and the 10 HCC827 cells captured on the beads prepared in Example 1 were mixed with 4 μL of a PBS buffer, added 3 μL of an alkali solution (400 mM KOH), and incubated at room temperature for 5 minutes. Then, the resultant mixture was neutralized by adding 3 μL of 10 mM Tris-HCl (pH8.3).

A first forward primer for the first qPCR, a first reverse primer for the first qPCR, a second forward primer for the second qPCR, and a second reverse primer for the second qPCR were used for the qPCR reactions, and the details are shown in Table 1 below.

TABLE 1

| qPCR | Primer | Nucleotide sequence |
|---|---|---|
| first | first forward | 5′-CTGGCATGAACATGACCCTG-3′ |
| qPCR | primer | (SEQ ID NO: 1) |
| | first reverse | 5′-CTGACCTAAAGCCACCTCCTT-3′ |
| | primer | (SEQ ID NO: 2) |
| second | second forward | 5′-AGCCAGGAACGTACTGGTGA-3′ |
| qPCR | primer | (SEQ ID NO: 3) |
| | second reverse | 5′-GCCTCCTTCTGCATGGTATT-3′ |
| | primer | (SEQ ID NO: 4) |

The incubated reactant or the reactant neutralized after the alkaline lysis, 250 nM of the first forward primer, and 250 nM of the first reverse primer, and qPCR Premix (Exiqon) were mixed together. The resulting mixture was subjected to the first qPCR, wherein the resulting mixture was incubated for 10 min at 95° C., and a cycle consisting of 10 seconds at 95° C., and 60 seconds at 63° C. was repeated 28 times.

A product of the first qPCR, 50 nM of the second forward primer, 50 nM of the second reverse primer, and qPCR Premix (Exiqon) were mixed together. The resulting mixture was subjected to the second qPCR, wherein the resulting mixture was incubated for 10 min at 95° C., and a cycle consisting of 10 seconds at 95° C., and 60 seconds at 63° C. was repeated 45 times.

qPCR was replicated 3 times, and a crossing point (Cp) for each qPCR was determined. The average Cp was calculated therefrom, and the result is shown in Table 2 below.

TABLE 2

| Sample | Method of lysis | Average Cp |
|---|---|---|
| 60 pg of | cell lysis buffer 2 | 10.7 |
| genomic DNA | cell lysis buffer 2 & proteinase K | 10.9 |
| 10 HCC827 cells | cell lysis buffer 2 | 20.3 |
| | cell lysis buffer 2 & proteinase K | 13.5 |
| | neutralization after alkaline lysis | 37.4 |
| 10 HCC827 cells | cell lysis buffer 2 & proteinase K | 35.7 |
| captured on beads | neutralization after alkaline lysis | 38.5 |

As shown in Table 2, the isolated cells treated with the cell lysis buffer 2 and proteinase K showed a relatively low Cp as a result of qPCR even in the absence of an additional purification step. In contrast, the HCC827 cells captured on the beads showed a relatively high Cp as a result of qPCR. Accordingly, it was confirmed that the HCC827 cells captured on the beads have a relatively low efficiency qPCR amplification when treated with the cell lysis buffer 2 and proteinase K.

### Example 3

### Effects of Sonication or Heat Treatment, Bead Separation, and Subsequent Treatment with Proteinase K on qPCR

3-1. Effects of Sonication, Beads Separation, and Subsequent Treatment with Proteinase K on qPCR

In order to use the HCC827 cells captured on the beads for qPCR without an additional purification step, the cells were sonicated, separated from the beads, and then treated with proteinase K.

The 10 HCC827 cells or 10 HCC827 cells captured in the beads prepared in Example 1 were added to the cell lysis buffer 2 (50 mM KCl, 1.5 mM MgCl₂, and 10 mM Tris-HCl (pH8.3)) or a cell lysis buffer 3 (100 mM KCl, 1.5 mM MgCl₂, and 10 mM Tris-HCl (pH8.3)) to a final volume of 10 μL. The mixture was sonicated at room temperature for 1 minute using a sonicator (sonorex super RK255H, BANDELIN). 10 μL of distilled water was added to the sonication reactant, beads were separated from the reactant by a magnet, and the reactant was recovered. 20 μL of the recovered reactant was either added or not added to 1 μg of proteinase K (Sigma) and then incubated at 55° C. for 1 hour. A sample not including cells was used as a negative control.

A qPCR was replicated 3 times on the incubated reactant as described in Example 2, and a Cp for each qPCR was determined therefrom. The determined Cps are shown in Table 3 below.

TABLE 3

| | Cell Lysis | Presence of | Cp | | |
|---|---|---|---|---|---|
| Sample | Buffer | proteinase K | qPCR 1 | qPCR 2 | qPCR 3 |
| Negative | cell lysis | — | 34.5 | | |
| control | buffer 3 | | | | |
| 10 cells | cell lysis | ∘ | 7.9 | 8.9 | 8.9 |
| | buffer 3 | | | | |
| 10 cells | cell lysis | ∘ | 11.3 | 10.5 | 10.1 |
| captured | buffer 3 | | | | |
| on beads | cell lysis | ∘ | 16.9 | 12.8 | 20.5 |
| | buffer 2 | | | | |

As shown in Table 3, the reactant, where the HCC827 cells captured on the beads were sonicated, separated from the beads, and then treated with proteinase K in the presence of the cell lysis buffer 3, showed a relatively low Cp, thus having an improved qPCR amplification efficiency. Accordingly, it was confirmed that the amplification efficiency of qPCR is improved when the cell captured on the beads is treated in the sequential order of sonication, beads separation, and subsequently with proteinase K.

3-2. Effect of Heat Treatment, Bead Separation, and Subsequent Treatment with Proteinase K on qPCR

qPCR was performed by treating the cell captured on the beads in the sequential order of heat treatment, beads separation, and subsequently with proteinase K, different from the method in Example 3-1.

The 10 cells captured on beads prepared in Example 1 were added the cell lysis buffer 3 (100 mM KCl, 1.5 mM MgCl₂, and 10 mM Tris-HCl (pH8.3)), sonicated at room temperature (RT) for 10 minutes, or incubated at 50° C. for 10 minutes, at 75° C. for 10 minutes, or at 99° C. for 10 minutes. The resultant was added with 10 μL of distilled water, beads were separated from the incubated sample by using a magnet, and the reactant was recovered therefrom. 20 μL of the recovered reactant was either added or not added with 1 μg of proteinase K (Sigma) and then incubated at 55° C. for 1 hour. A sample not including cells was used as a negative control.

PCR was replicated 3 times on the incubated reactant as described in Example 2, and a Cp for each qPCR was determined therefrom. The determined Cps are shown in Table 4 below.

TABLE 4

| | Treatment | | | | |
|---|---|---|---|---|---|
| | before bead | Presence of | Cp | | |
| Sample | separation | proteinase K | qPCR 1 | qPCR2 | qPCR3 |
| Negative | 10 min at RT | — | 37.0 | 39.6 | 36.6 |
| control | | | | | |
| 10 cells | sonication | ∘ | 11.0 | 9.6 | 10.3 |
| captured | 10 min at RT | ∘ | 21.6 | 18.3 | 36.6 |
| on beads | 10 min at 50° C. | ∘ | 35.8 | 21.5 | 37.9 |
| | 10 min at 75° C. | ∘ | 37.8 | 38.3 | 37.7 |
| | 10 min at 99° C. | ∘ | 18.9 | 38.0 | 36.6 |

As shown in Table 4, the reactant, where the cells captured on the beads were heat treated, separated from the beads, and then treated with proteinase K, showed a higher Cp than the reactant treated with sonication, thus having a lower rate of qPCR amplification.

### Example 4

### Confirmation of Reproducibility of Sonication, Bead Separation, and Subsequent Treatment with Proteinase K

As described in Example 3-1, the 10 cells captured on beads were sonicated, separated from the beads, and then treated with proteinase K, and then qPCR was performed thereon. qPCR was performed 4 times on three identical samples and a Cp for each qPCR was determined therefrom. The determined Cps are shown in Table 5 below.

TABLE 5

| | | Cp | | | |
|---|---|---|---|---|---|
| | | qPCR No. | | | |
| Sample | | qPCR 1 | qPCR 2 | qPCR 3 | qPCR 4 |
| Negative Control Group | | 34.5 | 37.7 | 35.0 | 32.8 |
| 10 cells | Replication No. 1 | 11.3 | 11.0 | 11.7 | 12.2 |
| captured | Replication No. 2 | 10.5 | 9.6 | 11.0 | 11.2 |
| on beads | Replication No. 3 | 10.1 | 10.3 | 12.2 | 10.5 |

| | | | | |
|---|---|---|---|---|
| Average Cp | 10.6 | 10.3 | 11.7 | 11.3 |
| Standard Deviation | 0.6 | 0.7 | 0.6 | 0.8 |

As shown in Table 5, the qPCRs performed after the sequential treatment of sonication, beads separation, and the subsequent treatment with proteinase K showed a relatively low standard deviation, thus having excellent reproducibility.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of amplifying a nucleic acid of a cell, the method comprising:
applying ultrasonic waves to lyse a cell bound to a solid support to provide a cell lysate comprising a nucleic acid;
separating the cell lysate from the solid support;
adding a protease to the separated cell lysate; and
amplifying the nucleic acid using a nucleic acid polymerase.

2. The method according to claim 1, wherein the cell is lysed in the presence of a solution containing a cation.

3. The method according to claim 2, wherein the cation is K⁺, Na⁺, Mg²⁺, or a combination thereof.

4. The method according to claim 2, wherein the concentration of the cation is from about 50 mM to about 200 mM.

5. The method according to claim 2, wherein the solution further comprises a non-ionic surfactant.

6. The method according to claim 5, wherein the non-ionic surfactant is polyoxyethylene glycol alkyl ether, polyoxypropylene glycol alkyl ether, glucoside alkyl ether, polyoxyethylene glycol octylphenol ether, polyoxyethylene glycol alkylphenol ether, glycerol alkyl ester, polysorbate, sorbitan alkyl ester, cocamide, dodecylmethylamine oxide, polyethoxylated tallow amine (POEA), or a combination thereof.

7. The method according to claim 1, wherein the cell is bound to the solid support via a material that specifically binds to the cell.

8. The method according to claim 7, wherein the material is a material that binds a protein, a substrate of an enzyme, a co-enzyme, a regulating factor, a material that binds a receptor, a lectin, a sugar, a glycoprotein, an antigen, an antibody, an antigen-binding fragment, a hormone, a neurotransmitter, phospholipid-binding protein, a protein including a pleckstrin homology (PH) domain, a cholesterol-binding protein, or a combination thereof.

9. The method according to claim 1, wherein the solid support is polystyrene, polypropylene, a magnetic particle, or a combination thereof.

10. The method according to claim 1, wherein the protease is serine protease, threonine protease, cysteine protease, aspartate protease, glutamic acid protease, metalloprotease, or a combination thereof.

11. The method according to claim 10, wherein the serine protease is proteinase K.

12. The method according to claim 1, wherein the protease is incubated with the cell lysate at about 15° C. to about 65° C.

13. The method according to claim 1, wherein the nucleic acid polymerase is DNA polymerase, RNA polymerase, reverse transcriptase, or a combination thereof.

14. The method according to claim 1, wherein the amplification is polymerase chain reaction (PCR), nucleic acid sequence-based amplification (NASBA), ligase chain reaction (LCR), strand displacement amplification (SDA), rolling circle amplification (RCA), or a combination thereof.

15. The method according to claim 1, wherein the nucleic acid amplification is whole genomic nucleic acid amplification or targeted nucleic acid amplification.
